(19) United States
(12) Patent Application Publication
(10) Pub. No.: US 2002/0148003 A1
(43) Pub. Date: Oct. 10, 2002

(54) Transgenic plant or plants with a naturally high water content overproducing at least two amino acids of the aspartate family

(75) Inventors: Ingrid Maria Van Der Meer, Amsterdam (NL); Oscar Frederik Jozef Vorst, Utrehct (NL); Peter Martin Bruinenberg, Hoogezand (NL); Johannes Pieter Marinus Sanders, Groningen (NL); Adrianus Johannes Van Tunen, Wageningen (NL)

Correspondence Address: YOUNG & THOMPSON, 745 SOUTH 23RD STREET 2ND FLOOR, ARLINGTON, VA, 22202

(73) Assignee: AVEBE, FOXHOL (NL)

(21) Appl. No.: 10/155,970

(22) Filed: May 29, 2002

Related U.S. Application Data
(63) Continuation of application No. 09/308,395, filed on Jun. 7, 1999, now abandoned.
Application No. 09/308,395 is a 371 of international application No. PCT/NL96/00458, filed on Nov. 18, 1996.

(51) Int. Cl. A01H 5/00; C12N 5/04; C12N 15/82

(52) U.S. Cl. 800/278; 435/320.1; 435/419

(57) ABSTRACT

A chimeric double gene construct comprising a nucleic acid sequence encoding an enzyme having aspartate kinase (AK) activity and a nucleic acid sequence encoding an enzyme having dihydrodipicolinate synthase (DHPS) activity is provided. This construct is capable of differential expression of the two genes resulting in an increased level of both lysine and threonine more than 5-fold the wild type level of each amino acid in a plant or parts thereof.

### DESCRIPTION

### SUMMARY OF THE INVENTION

A chimeric double gene construct comprising a nucleic acid sequence encoding an enzyme having aspartate kinase (AX) activity and a nucleic acid sequence encoding an enzyme having dihydrodipicolinate synthase (DHPS) activity is provided. This construct is capable of differential expression of the two genes resulting in an increased level of both lysine and threonine more than 5-fold the wild type level of each amino acid in a plant or parts thereof. This construct also renders increase in the methionine level possible. This expression is now possible without incurring the previous problems associated with high accumulation of lysine or combined expression of both AK and DHPS genes in a plant. The expression regulation should be such that expression occurs in such a, way that both lysine and threonine are produced to a comparable extent without damaging the plant i.e. without causing a negative abberations in the phenotype compared to wild type plants. The construct resulting in lysine and threonine increase also provides an increase in the methionine level.

### BACKGROUND OF THE INVENTION

Human and monogastric animals cannot synthesize 9 out of 20 amino acids and therefore need to obtain these essential amino acids from their diet. The diet of human beings and livestock is based largely on plant material. Among the essential amino acids needed for animal and human nutrition are lysine and threonine. These essential amino acids are often only present in low concentrations in crop plants. Therefore, synthetic amino acids are usually added as supplements to grain-based and other vegetable based diets, in order to increase the nutritional value of feed.

Various attempts have been made in the past to increase the levels of free threonine and lysine in plants by classical breeding and by mutant selection, but with little success. Also it has been attempted to increase both lysine and threonine simultaneously in the same transgenic plant by molecular genetic modification. Increase of free lysine proved to operate at the expense of the accumulation of free threonine (Shaul and Galili, 1993; Falco et al., 1995). To date no description of increased methionine level has been given.

### Biosynthesis of Aspartate-Family Amino Acids

The essential amino acids lysine, threonine and methionine are synthesized from aspartate by a complex pathway which is similar for bacteria and higher plants (FIG. 1). The aspartate family pathway has been characterized in detail in Escherichia colt by isolation of enzymes involved in the pathway, which were later also purified from higher plants (Bryan, 1980. Umbarger, 1978).

The rate of synthesis of the aspartate-family amino acids is regulated primarily by a complex process of feedback inhibition of the activity of some key enzymes in the pathway by the relevant amino acid end product. The first enzymatic activity in the pathway that is common to all of the aspartate-family amino acids, aspartate kinase (AK) activity, is feedback inhibited by both lysine and threonine. In addition, lysine also inhibits the activity of the enzyme dihydrodipicolinate synthase (DIPS), the first enzyme of the pathway after the branch point that leads to the synthesis of lysine. Threonine inhibits the activity of homoserine dehydrogenase (HSD), the first enzyme involved in the biosynthesis of threonine (Matthews et al., 1989).

The enzyme aspartate kinase (AK) catalyses the phosphorylation of aspartate to form 3-aspartyl phosphate, with the accompanying hydrolysis of ATP. Both in E. coli and in plants several different AK isoenzymes have been identified which are differentially inhibited either by lysine or by threonine. AK-III, the product of the E. coli lysC locus has been shown to consist of two identical subunits as a homodimer (Cassan et al., 1986; Richaud et al., 1973). The E. coli LysC gene has been cloned and sequenced (Cassan et al., 1986).

The product of AK activity, 3-aspartyl phosphate, is converted in the next enzymatic step to 3-aspartic semialdehyde (3-ASA), which serves as a common substrate for the synthesis of both lysine and threonine. The enzyme dihydrodipicolinate synthase (DHPS) catalyses the first reaction that is unique to lysine biosynthesis, the condensation of 3-aspartate semialdehyde with pyruvate to form 2,3-dihdrodipicolinate. In E. coli this enzyme is encoded by the dapA locus and appears to consist of four identical subunits as a homotetramer (Shedlarski and Gilvarg, 1970). The E. coli dapA gene has been cloned and sequenced (Richaud et al., 1986). In plants DHPS enzyme also appears to be a single enzyme comparable to the E. coli enzyme. Among the major reguIatory enzymes of the aspartate family pathway in plants. DHPS is the most sensitive to feedback inhibition by its end product (I₅₀ of DHPS for lysine ranges between 10 and 50 μM). Plant DHPS is about 10-fold more sensitive to lysine inhibition than are plant AKs that are sensitive to lysine (I₅₀ between 100 and 700 μM). Plant DHPS is about 100-fold more sensitive to lysine inhibition than E. coli DHPS (I₅₀ is about 1 mM) (Yugari and Gilvarg, 1962: Galili, 1995)

Homoserine dehydrogenase (HSD) catalyses the first reaction that is specific for the synthesis of threonine, methionine and isoleucine. Higher plants generally possess at least two forms of HSD: a threonine-sensitive form and an insensitive form (Bryan, 1980. Lea et al., 1985).

Several lines of evidence have indicated that in plants AK is the rate-limiting enzyme for threonine synthesis, while DHPS is the major rate-limiting enzyme for lysine synthesis. Mutants of several plant species possessing feedback-insensitive AK isozyme were found to overproduce free threonine, but exhibited only a slight increase in the level of lysine (Bright et al., 1982; Cattoir-Reynaerts et al., 1983; Dotson et al., 1990; Frankard et al., 1992). On the other hand, a feedback-insensitive DHPS mutant tobacco plant overproduced lysine (Negrutiu et al., 1984). Similar results have been reported with transgenic plants expressing feedback-insensitive DHPS or AK from E. coli (Glassman, 1992; Perl et al., 1992; Shaul and Galili, 1992 a and b). Transgenic plants that expressed the E. coli AK overproduced threonine and exhibited only a slight increase in the level of lysine. Studies of transgenic plants have also demonstrated that AK and DHPS are not only regulated by feedback inhibition, but that the levels of these enzymes also limit the rate of production of threonine and lysine. In transgenic plants a significant positive correlation was detected between the levels of the bacterial AK and DHPS enzymes and the levels of free threonine and lysine, respectively (Shaul and Galili, 1992 a and b).

However. transgenic plants expressing both a feedback-insensitive AK and a feedback-insensitive DHPS (Shaul and Galili, 1993) contain free lysine levels that far exceed those in plants expressing only the introduced insensitive AK or DHPS. This lysine increase is also accompanied by a significant reduction in threonine accumulation as compared with transgenic plants expressing the insensitive AK only. This means that when DHPS becomes deregulated the branch leading to lysine synthesis competes strongly with the other branch of the pathway and a considerable amount of 3-aspartic semialdehyde is thus converted into lysine at the expense of threonine. The same results were obtained by crossing a lysine overproducing mutant with a threonine overproducing mutant characterized by an altered regulation of respectively DHPS and AK (Frankard et al., 1992).

European patent application No. 485.970 discloses a method of increasing the level of both free threonine and lysine by introducing into plants cells a first chimeric gene comprising a DNA sequence coding for an enzyme having AK activity and a second chimeric gene comprising a DNA sequence coding for an enzyme having DHPS activity. Both chimeric genes further comprise a DNA sequence enabling expression of the enzymes in the plant cells and subsequent targeting of the enzymes to the chloroplast.

European patent application No. EP 93908395 describes two isolated DNA fragments comprising a fragment encoding AK insensitive to inhibition by lysine and a second fragment encoding DHPS which is at least 20-fold less sensitive to inhibition by lysine than plant DHPS. It is claimed that the lysine-insensitive AK causes a higher than normal threonine production and that the DHPS causes a higher than normal lysine production in transformed plants.

However, while it is shown that transgenic plants expressing feedback-insensitive DHPS from E. coli overproduce lysine and that transgenic plants expressing the E. coli AK overproduce threonine (Glassman. 1992; Perl et al., 1992; Shaul and Galili, 1992 a and b), combining these two genes in transgenic plants never resulted in a comparable increase of both threonine and lysine. Shaul and Galili (1993) obtained transgenic plants expressing both feedback-insensitive AK and feedback-insensitive DHPS by crossing transgenic plants that expressed each of these enzymes individually. These plants were shown to contain free lysine levels that far exceeded those in plants expressing only the insensitive DHPS. The lysine increase however was accompanied by a significant reduction in threonine accumulation as compared with plants expressing the insensitive AK only.

The same results were found when feedback-insensitive bacterial DHPS and AK enzymes encoded by the Corynebacterium dapA gene and a mutant E. coli lysC gene, respectively, were expressed together in transgenic canola and soybean seeds. Several hundred-fold increases in free lysine in transgenic seed was observed, whereas the accumulation of excess threonine that was seen in transgenic seed expressing feedback-insensitive AK alone was prevented by the co-expression of DHPS (Falco et al., 1995).

Furthermore, abnormal phenotypes have been observed in transgenic plants in which the free lysine concentration is increased in the whole plant to more than 10-fold the concentration found in untransformed plants (Shaul and Galili, 1992, Glassman, 1992; Frankard et al., 1992).

In European patent application No. EP 435970 the expression of both feedback-insensitive AK and DHPS genes was driven by the same constitutive CaMV35S promoter. Also in the experiments of Galili and Shaul (1993) and of Falco et al. (1995) in which both feedback-insensitive AK and DHPS were co-expressed in transformed plants, the same promoter was used to drive expression of the two genes.

The problems that are encountered when using genetic engineering technology to increase both free lysine and threonine in plants are reported by Falco et al. (1995). They also showed that using the same seed-specific promoter to drive both feedback-insensitive AK and DHPS expression only resulted in increasing the lysine content of canola and soybean seeds. Furthermore, they showed that seeds from transformants accumulating the highest levels of lysine had en abnormal appearance and germinated poorly (Falco et al. (1995).

The following hypothesis is offered on the basis of the findings from the prior articles. The ratio between lysine and threonine synthesis in plants is regulated by at least two factors: First, the availability of 3-ASA which is the common substrate for the two key-enzymes specific for threonine and lysine synthesis (respectively homoserine dehydrogenase and DHPS). Second, the competition between these two key-enzymes for 3-ASA as a common substrate. The level of 3-ASA seems to be determined by the activity of AK. When feedback-insensitive AK is overexpressed in transgenic plants, the higher 3-ASA concentration can be channeled into the threonine synthesis branch which may also result in an increase of methionine. However, when DHPS becomes feedback-insensitive the branch leading to lysine synthesis competes strongly with the other branch of the pathway and a considerable amount of 3-ASA is thus converted into lysine at the expense of threonine synthesis. In order to increase the amount of both free lysine and threonine in transgenic plants to desirable levels, the timing and the level of expression of each introduced feedback-insensitive gene should be highly regulated. The production of methionine another amino acid of the aspartate pathway is also concomitantly increased.

According to the invention the expression of a nucleic acid sequence encoding an enzyme having aspartate kinase (AK) activity and a nucleic acid sequence encoding an enzyme having dihydrodipicolinate synthase (DHPS) activity is regulated such that the expression of the two sequences is different in strength and such that the expression of the AK encoding sequence occurs at a higher rate than that of the DHPS encoding sequence. This can be achieved by using different strength promoters or even by using the same promoters but additionally providing the DHPS regulating promoter with an enhancer. The object of the invention is to generate plants that overproduce both threonine and lysine to a comparable extent. Overproduction of methionine is also an objective. This can occur through using special sequences according to the invention with recombinant DNA technology techniques that are well known in the art such as by transformation of plant cells.

Preferably the expression of the DHPS and AK encoding sequences is regulated at different stages of plant development, so that the plant has a chance to develop and mature without interference from overproduced free amino acid, in particular overproduced lysine. This can be achieved in a number of manners.

Firstly by regulating at least the DHPS encoding sequence with an inducible promoter which inducible promoter can be induced after the AK encoding sequence has begun to be expressed. The AR encoding sequence may naturally also be regulated by an inducible promoter, however with the restriction that a stronger expressing system is provided for the AK encoding sequence than for the DHPS encoding sequence and the restriction that the expression of DHPS occurs at a later stage than that of the AK encoding sequence. In particular it is preferred to induce the DHPS encoding sequence when the plant has reached a sufficient degree of maturity not to be negatively influenced by overproduction of lysine.

Secondly the expression at different stages can be achieved in an alternative embodiment of the invention by regulating expression with promoters that are associated with cell types or organs and drive natural expression at different stages of plant development. Suitable examples of promoters for the DHPS encoding sequence are promoters associated with processes that occur late in plant development or even upon maturity. In particular tuber or fruit associated promoters are suited for regulating expression of the DHPS encoding sequence. The AK encoding sequence may also be regulated by such promoters but with the restriction that a stronger expressing system is provided for the AK encoding sequence than for the DHPS encoding sequence. Preferably in such an embodiment also the DHPS encoding sequence is expressed later than the AK encoding sequence for example due to the use of an inducible promoter.

A very strong constitutive promoter can be used to drive feedback-insensitive AK expression throughout the whole plant during all stages of organ developments, resulting in a higher 3-ASA concentration which can be channeled into the threonine synthesis branch and will lead to threonine and methionine overproduction. In combination therewith the feedback-insensitive DHPS enzyme can be regulated such that it is only expressed at a lower level and at a later stage e.g. in specific plant organs. The expression of the AK encoding sequence can be constitutive but it may also be restricted to specific organs.

The AK encoding sequence may be expressed in the name organs as the DHPS encoding sequence, however the encoding sequences must be differentially expressed. Because the expression of DHPS is at a later stage and at a lower level, the branch leading to lysine synthesis competes only weakly and during a well defined period of development with the other branch of the pathway and both threonine and lysine will be overproduced. Methionine also a component of the aspartate pathway is also overproduced.

It is preferable when very high levels of lysine are to be achieved. e.g. tenfold or more higher than normal that the DHPS encoding sequence is expressed in specifically targeted organs to prevent negative characteristics of the phenotype from appearing in the plant. Such targeted plant organs are organs that develop later on in plant maturation such as tubers, seed, fruit and flowers. Because lysine is only overproduced in the specifically targeted plant organs like tubers, the rest of the plant will develop normally without showing aberrant phenotype.

The expression of the DHPS encoding sequence is preferably carried out in specific plant organs with a high water content. This also offers the advantage that minimum damage to phenotype will occur. Such organs comprise tubers, fruit, flowers or leaves. Preferably water rich organs comprise more than 70% water,preferably more than 80%. In addition the expression of the DHPS encoding sequence in a plant organ with a high water content provides the required amino acids readily dissolved in a water fraction which renders it easily accessible for harvesting. For this reason it is also preferable for the AK encoding gene also to be at least expressed in a plant organ with a high water content. Most preferably the two genes should be expressed with an area of overlap of expression being the plant organ with a high water content in which the DHPS encoding sequence is expressed. Preferably the water rich organ will be a readily harvestable organ such as tuber, flower, fruit, root or leaves.

Such an organ can either be used as such as source of nutrition comprising a high content of at least two amino acids of the aspartate family selected from methionine, lysine and threonine or can be subjected to an extraction process for obtaining a water fraction, said water fraction comprising a high content of at least two amino acids of the aspartate family selected from methionine, lysine and threonine. Naturally the plant as such or a part of plant comprising the water rich organ can be harvested and used as such e.g. as food or food supplement and/or be subjected to a water extraction process. Such water fraction obtained from a plant or plant organ according to the invention can subsequently also be used as such for nutritional purposes. Numerous simple economical extraction processes are known e.g. from fruit or vegetable juice production. It thus becomes possible to provide fruit or vegetable juices in a manner known per se, said fruit or vegetable juices however being of considerable additional nutritional value due to the high presence of at least two amino acids of the aspartate family selected from methionine, lysine and threonine.

Plants or parts of plants e.g. fruit, flowers, tubers, roots or leaves according to the invention as such are particularly suited for vegetarians due to their automatic supplementation of two essential amino acids. In addition the water fraction from plants or parts of plants comprising the water rich organs expressing the combination of genes according to the invention provides an easily obtainable source with a high content of at least two amino acids of the aspartate family selected from methionine, lysine and threonine from which the over produced amino acids can readily be obtained in large amounts. The increased levels of methionine result in an easily obtainable source of methionine for the first time. It now becomes possible to obtain methionine, lysine and threonine in a simple manner.

The present invention relates to a chimeric double gene construct comprising

(a) a nucleic acid sequence encoding an enzyme having aspartate kinase (AK) activity,

(b) a nucleic acid sequence encoding an enzyme having dihydrodipicolinate synthase (DHPS) activity,

(c) an expression regulating sequence for the encoding sequence (a) operably linked to (a),

(d) an expression regulating sequence for the encoding sequence (b) operably linked to (b), said expression regulating sequences (c) and (d) being such that the expression of the two sequences is different in strength and such that the expression of the AK encode sequence (a) occurs at a higher rate than that of the DHPS encoding sequence (b) i.e. (c) is stronger than (d). In addition the sequences (a) and (b) of the chimeric double gene construct must also each comprise a nucleic acid sequence (e) coding for a transit peptide which is involved in the translocation of protein from the cytosol into the plastids (Van den Broek et al., 1985; Schreier et al., 1985) as the organelle in which lysine and most of the threonine biosynthesis takes place in higher plants is the plastid. This nucleic acid sequence encoding a chloroplast transit peptide fused to the encoding sequences of (a) and (b), will on expression produce a fused AK/transit peptide or DHPS/transit peptide chimeric protein in the cytoplasm of the transformed plant cell, which will be transported to the plastids, where increased production of both threonine and lysine is thereby obtained. Nucleic acid sequences coding for any kind of plastid transit peptide can be used in this invention. A suitable example is provided by the DNA sequences derived from the ferredoxin gene coding for targeting of proteins into the chloroplast stroma (Smeekens et al., 1985a) and sequences from the plastocyanine gene coding for targeting of proteins into the chioroplast lumen (Smeekens et al., 1985b). The preferred DNA sequence coding for plasmid targeting of AK and DHPS encodes the transit peptide originating from the pea rbcS-3A gene (FIG. 2; Fluhr et al., 1986). The 3′ end of the nucleic acid sequence coding for the transit peptide is fused to the sequence encoding AK or DHPS.

The sequences encoding AK and DHPS (a) and (b) must in turn also be fused to a transcription termination DNA signal (g) and (h) respectively. This termination signal comprises a 3′ transcription termination and a mRNA polyadenylation signal. Termination signals present at the 3′ flanking region of any cloned gene can be used, e.g. from the pea rbcS gene, the bean phaseoline gene, or the nopaline synthase gene derived from the Ti plasmid of Agrobacterium tumefaciens. The preferred terminator sequence originates from the 3′ flanking region of the octopine synthase gene from the Ti plasmid of Agrobacterium tumefaciens (FIG. 2; Greve et al., 1983).

The gene construct according to the invention preferably comprises as expression regulating sequence (d) for nucleic acid sequence (b) a regulating sequence enabling expression of DHPS at a later stage during development compared to the expression of nucleic acid sequence (a). In an alternative embodiment the expression regulating sequence (d) is a sequence that does not lead to constitutive expression. Preferably the sequence leads to expression in one or more specific plant organs e.g. a tuber, flower, fruit, root or stem. A preference exists for the sequence to regulate expression in water rich organs.

The expression regulating sequence (c) for nucleic acid sequence (a) can be a sequence that enables high expression of the encoding sequence (a) at an early stage during plant and/or plant organs development. The expression regulating sequence (c) for nucleic acid sequence (a) can be a sequence that enables high expression of the encoding sequence (a) in all plant cells or plant organs. The expression regulating sequence (c) is suitably a constitutive promoter. Such a constitutive promoter can be the cauliflower mosaic virus (CaMV) 35S promoter.

The expression regulating sequence (c) can comprise not only a promoter but also an expression enhancer sequence. If this is the case the expression regulating sequence (d) can comprise the same promoter or a promoter of the same strength as that of expression regulating sequence (c) as the expression level of encoding sequences (a) and (b) will be different.

The expression regulating sequences in particular the promoters are to be found in the 5′ region of each of the encoding sequences (a) and (b). At the 3′ end of the promoter a short nucleic acid sequence representing 5′ mRNA non-translated sequence may be added, which enhances translation of the mRNA transcribed from the chimeric genes. An example is the omega sequence derived from the coat protein gene of the tobacco mosaic virus (Gallie et al., 1987), of the alfalfa mosaic virus translational enhancer (Brederode et al., 1980).

A suitable enhanced promoter for expression regulating sequence (c) to drive expression of the AK enzyme encoding sequence (a) according to the invention is the enhanced cauliflower mosaic virus (CaMV) 35S promoter (Benfey et al., 1990; Pen et al., 1992). In this promoter the enhancer sequence is duplicated which turns it into a strong promoter which drives expression constitutively throughout the whole plant in all stages of development. Any other promoter which drives constitutive expression at high level can be used.

The promoters for expression regulating sequences (c) and (d) may originate from either mono- or dicotyledonous plants. They are preferably tissue specific but can also be non-tissue specific promoters. The same tissue-specific promoter in expression regulating sequence (d) that regulates DHPS (b) expression can be used to drive feedback-insensitive AK expression (a) when fused to an enhancer.

A suitable organ specific regulating sequence (d) to drive expression of the DHPS enzyme encoding sequence (b) according to the invention is the tuber-specific class I patatin promoter from Solanum tuberosum (Mignery et al., 1988; Wenzler et al., 1989). Other tuber-specific promoters can be used to drive the DHPS expression like the proteinase inhibitor II promoter from potato (Keil et al., 1989), or the cathepsin D inhibitor potato promoter (Herbers et al., 1994). Also other tissue-specific promoters can be used like the fruit-specific promoter from tomato polygalacturonidase gene (Grierson et al., 1986), the seed-specific phaseoline promoter from been (Sengupta-Gopalan, 1985) or the root specific CaMV 35S promoter subdomains (Benfey et al. 1990).

The expression regulating sequences (c) and (d) can both comprise inducible promoters. Preferably the induction stimulus will be different for the two promoters in order to allow differential expression of the encoding sequences to which they are operably linked. Other examples of inducible promoters are the light inducible promoter derived from the pea rbcS gene (Coruzzi et al., 1984) and the actine promoter from rice (McElroy et al., 1990).

The nucleic acid encoding sequences (a) and (b) may be derived from any suitable source, e.g. from different plant cells or from bacteria, or may be a synthetic encoding sequence. In a preferred embodiment, such a sequence (a) encodes an enzyme that is less sensitive to inhibition by lysine and such a sequence (b) encodes an enzyme that is less susceptible to inhibition by threonine than the corresponding wild type plant enzymes. Suitably such sequences are derived from endogenous organisms. The preferable exogenous sources are bacteria, e.g. E. coli. A suitable nucleic acid sequence (a) encoding AK activity is the DNA sequence of E. coli mutant LysC gene coding for the isoenzyme AK-III, which is less sensitive to lysine than the plant enzyme (Boy et al. 1979; Cassan et al., 1983). A suitable nucleic acid sequence (b) coding for a DHPS enzyme is the dapA gene from E. coli (Richaud et al., 1983). The resulting expression product is less sensitive to lysine inhibition than the plant DHPS enzyme. Naturally sequences encoding mutated plant enzymes which are less sensitive to feedback inhibition are also suitable embodiments of sequences that can be used.

By way of example the chimeric double gene construct can thus consist of two encoding sequences encoding AK and DHPS driven by different promoters, both present in the same binary vector (FIG. 2). The AK encoding part of the chimeric gene construct has the strong (enhanced) cauliflower mosaic virus 35S promoter in the 5′ region linked to the 5′ end of the omega DNA sequence; the omega DNA sequence is linked to the 5′ end of the DNA sequence coding for the chloroplast transit peptide derived from the pea rbcS-3A gene, which is linked to the 5′ end of the coding sequence of the mutant E. coli lysC allele coding for a desensitized AK-III, which is linked to the 5′ end of the octopine synthase terminator sequence.

The DHPS encoding part of the chimeric gene construct has basically the same structure, but the strong constitutive promoter of the AK gene construct is replaced by a weaker tuber-specific potato patatin (class I) promoter and the lysC gene coding for desensitized AK-III is replaced by the E. coli dapA gene coding for DHPS.

The invention also relates to an expression vector comprising a chimeric double gene construct of the invention in any of the embodiments described.

Transgenic plants containing said double gene construct in their cells also fall within the scope of the invention as do parts of such transgenic plants.

Preferred plants are those with a high water content either in the plant as such or plant parts with high water content: in the tubers, leaves, stems, fruit, roots or flowers. Suitable plants are potato plants, sugar beet plants, grass and plants that are used to provide fruit or vegetables for fruit juice or vegetable juice. Examples of suitable fruit are apple, orange, grapefruit, grape, tomato, mango, banana, melon, strawberry, cherry, kiwi. Examples of suitable vegetables are potato and sugarbeet. Any other fruit or vegetable comprising a similar amount of water is considered suitable.

Preferably the plant or plant part will be derived from a plant or plant part normally considered acceptable for consumption in order to produce lysine and threonine destined for consumption. For economical reasons the plants that provide the highest amount of water fraction per acreage per unit of time at the most economical price for obtaining the water fraction are preferred. Naturally this will vary per country due to geographical and climatic influences. The plant can be a plant already commercially grown or can be a type of plant not commercial hitherto that can become commercially interesting upon incorporation of the gene construct according to the invention.

Parts of plants comprising water rich organs capable of expressing the double gene construct according to the invention form a preferred embodiment for plant parts as are plant cells that can develop into such water rich organs for plant cells. Preferred plant parts are also the water rich organs as such. Suitable organs are flowers, tubers and fruit and examples have already been discussed elsewhere in this document.

Plants or plant cells according to the invention produce concomitantly high levels of threonine and lysine. Plants according to the invention or plant cells according to the invention overproduce methionine. Preferably concomitantly at least two amino acids of the aspartate family are overproduced. Preferably in plants or plant parts according to the invention the lysine is produced only in specific organs with a high water content. For plants, plant parts or plant cells according to the invention expressing more than ten fold the amount of free lysine than the wild type preferably the DHPS encoding sequence is only expressed in particular late developing organs such as tubers, seed, fruit end flowers. Plant tissue derived from transgenic plants or plant cells according to the invention also falls within the scope of the invention.

In another embodiment, the invention provides transgenic plant cells containing or transformed by an expression vector according to the invention.

Both chimeric gene constructs can be subcloned into expression vectors, such as the Ti plasmids of Agrobacterium tumefaciens, the preferred plasmid being the binary vector pBINPLUS (Van Engelen et al., 1995).

The expression vector in which the double gene construct is cloned, is then introduced into plant cells. The introduction can be realised using any kind of transformation protocol capable of transferring DNA to either monocytoledonous or dicotyledonous plant cells. For example: transformation of plant cells by direct DNA transfer via electroporation (Dekeyser et al., 1990), via PEG precipitation (Hayashimoto et al., 1990) or via particle bombardment (Gordon-Kann et al., 1990) and DNA transfer to plant cells Via Section with Agrobacterium. The preferred method is via infection of plant cells with Agrobacterium tumefaciens (Horsch et al., 1985: Visser,1991). The methodology used here in the Example for potato or sugar beet can be used to improve crop plants of the type discussed above like tomato and apple.

Transformed plants are then selected by resistance to kanamycin or other antibiotics like hygromycin. Selection for plant cells or plants that overproduce any of threonine, lysine and methionine may also be performed by adding any of threonine, lysine and methionine to the plant growth media.

Plants containing in their cells both the AK and DHPS components of the gene construct according to the invention can also be obtained by crossing a transgenic plant containing only the AK gene construct (a) with a transgenic plant containing only the DHPS gene construct (b) with the corresponding expression regulating sequences (c) and (d) respectively. The separate gene constructs may optionally include the termination sequences and/or transit sequences as described above for the chimeric double gene construct.

The examples illustrate the invention but are not to considered the only embodiments. The content of the cited prior art is incorporated by reference.

### FIGURE DESCRIPTION

FIG. 1: A diagram of the aspartate family biosynthetic pathway. Only the major key enzymes and their products are indicated. Curved arrows represent feedback inhibition by the and product amino acids. DHPS, dihydrodipicolinate synthase; HDS, homoserine dehydrogenase; TDH, threonine dehydratase.

FIG. 2A=construct pAAP30

FIG. 2B=construct pAAP31

FIG. 2C=construct pAAP50

FIG. 2D=construct pAAP60

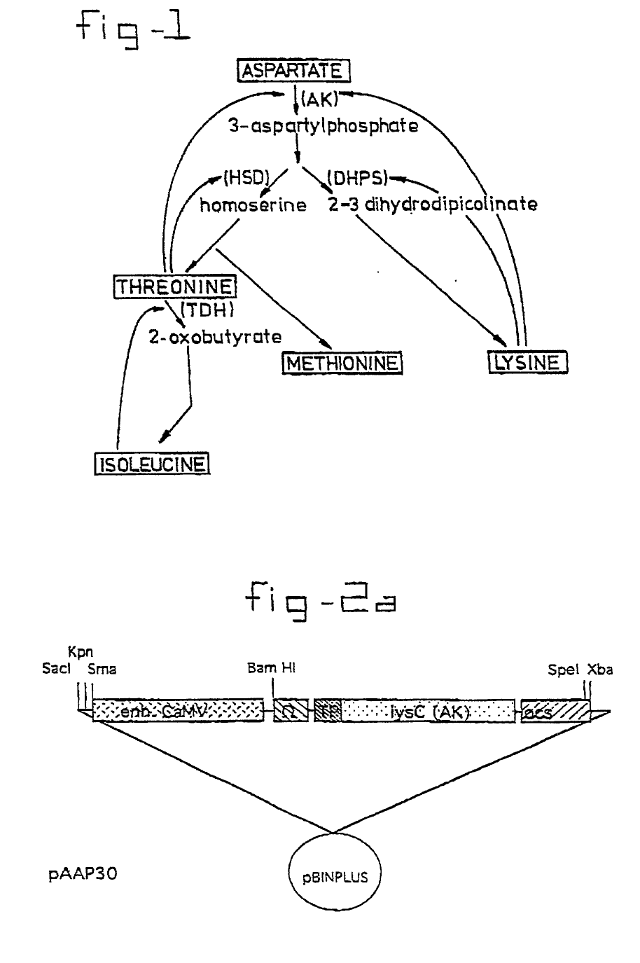

### EXAMPLE 1

### Construction of the Chimeric Double Gene Construct for Expression of AK and DHPS

DNA Isolation, subcloning, restriction analysis and DNA sequence analysis was performed using standard methods (Sambrook et al., 1989; Ausubel at al., 1994).

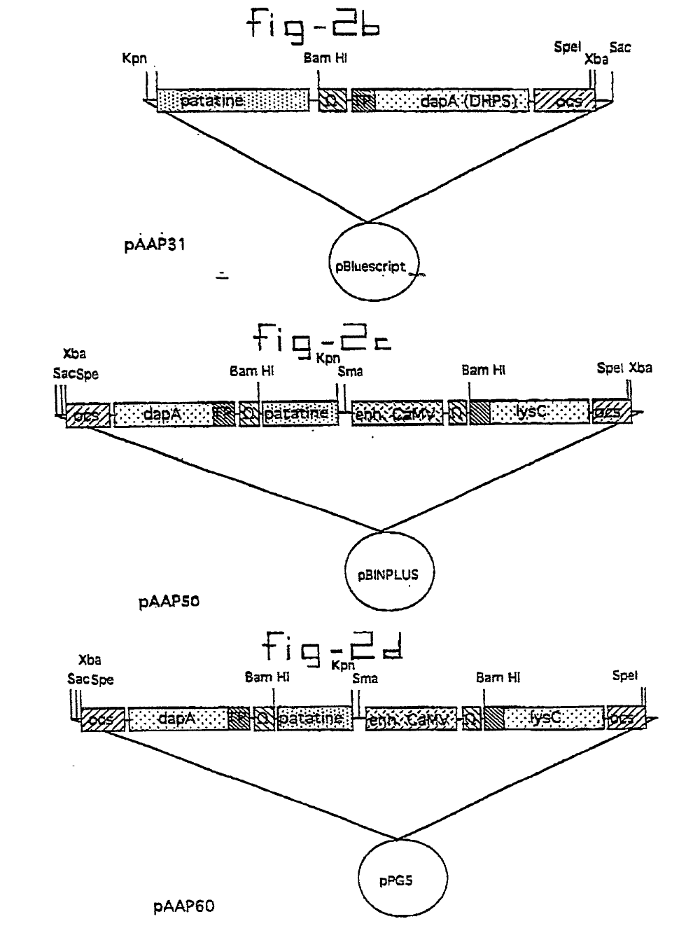

The chimeric gene containing the lysC gene was constructed by fusing the enhanced CaMV35S promoter to the chimeric lysC gene. This chimeric lysC construct contains the DNA fragment of the mutant lysC allele which is fused at the 5′ end to the Ω DNA sequence from the coat protein of tobacco mosaic virus (Gallie et al., 1989). Downstream of the lysC sequences the termination signal of the octopine synthase gene from Agrobacterium tumefaciens is inserted (Greve et al., 1983). A DNA fragment containing the pea rbcS-3A transit peptide coding sequence (Fluhr et al., 1986) was inserted between the Ω DNA and the lysC coding sequence. The chimeric lysC gene construct was cloned as a BamHI/SpeI fragment in pBluescript (Stratagen). The enhanced CaMV35S promoter (Benfey et al. 1990) was ligated as a SmaI/BamHI fragment in front of the lysC chimeric gene digested with BamHI/XbaI in the XbaI/SmaI fragment of the binary vector pBINPLUS (Van Engelen et al., 1995) (pAAP30; FIG. 2A).

The chimeric gene containing the DapA gene was constructed by fusing the enhanced CaMV35S promoter to the chimeric DapA gene. This chimeric DapA construct contains the DNA fragment of the E. coli DapA gene (Richaud et al. 1986) which is fused at the 5′ end to the Ω DNA sequence from the coat protein of tobacco mosaic virus (Gallie et al., 1989). Downstream of the DapA sequences the termination signal of the octopine synthase gene from Agrobacterium tumefaciens is inserted (Greve et al., 1983). A DNA fragment containing the pea rbcS-3A transit peptide coding sequence (Fluhr et al., 1986) was inserted between the Ω0 DNA and the DapA coding sequence. The chimeric DapA gene construct was cloned as a BamHI/SpeI fragment in pBluescript (Stratagen). The patatin promoter (Wenzler et al., 1989) was ligated as a blunt (HindIII filled in)/BamHI fragment in front of the DapA chimeric gene digested with SmaI/BamHI (pAAP31; FIG. 2B).

The double gene construct was realized by ligating the patatin-dapA chimeric gene as a KpnI/SacI fragment from pAAP31 into the binary vector with enh. CaMV35S-LysC (pAAP30) digested with SacI/KpnI (pAAP50; FIG. 2C).

### EXAMPLE 2

### Introduction of the Chimeric Genes into Potato

2.1 Transformation of Potato Plants

The binary vector pAAP50 was used for freeze-thaw transformation of Agrobacterium tumefaciens strain AGLO (Höfgen and Willmitzer, 1988). Transformed AGLO was subsequently used for inoculation of diploid potato (Solanum tuberosum, variety Kardal) stem explants as described by Visser (1991). After shoot and root regeneration on kanamycin-containing media plants were put in soil and transferred to the greenhouse. Plants regenerated (on kanamycin-free media) from stem explants treated with the Agrobacterium strain AGLO lacking a binary vector served as a control.

2.2 In Vitro Tuber Formation

In order to induce in vitro tuberization, nodal cuttings (about 4-5 cm long) of transformed potato plantlets were placed vertically in solid Murashige and Skoog (1962) medium supplemented with 10% (w/v) sucrose. 5 μM BAP. The cultures were maintained at 19° C. in the dark. After 14 days microtubers of 4 mm in diameter were harvested and analyzed for free lysine and threonine concent, and for AK and DHPS activity.

### EXAMPLE 3

### Analysis of Free Lysine, Threonine and Methionine Content, in Transgenic Potato Plant

Tissue (0.5-1.0 gram) was homogenized with mortar and pestle in 2 ml 50 mM Pi-buffer (pH 7.0) containing 1 mM dithiothreitol. Nor-leucine was added as an internal standard. Free amino acids were partly purified by extraction with 5 ml of a water:chloroform;methanol mixture (3:5:12). Water phase was collected and the remaining re-extracted twice. After concentration by lyophilization to 3 ml, a 20 μl sample was used in a 6-aminoquinolyl-N-hydroxysuccinimidyl carbamate (AQC) derivatization reaction. Derivitized amino acids were analyzed by DHPS on a reversed phase C18 column according to the manufactures instructions (Waters AccQ.Tag system).

### EXAMPLE 4

### Analysis of AK Activity in Trangenic Potato Plants

Microtubers, leaves or mature tubers were harvested and homogenized with mortar and pestle in an equal volume of cold 20 mM potassium-phosphate buffer. pH 7.0, containing 30 mM β-mercaptoethanol, 0.1 mM L-lysine, 0.1 mM L-threonine, 1 mM phenylmethylsulfonylfluoride and 0.5 μg/ml leupeptin. Following 5 min of centrifugation (16,000 g. 4° C.), the supernatant was collected and its protein concentration determined. Equal amount of protein were then tested for AK activity as described by Black and Wright (1955). The assay mixture (final volume of −0.25 ml) contained 300 μg of protein, 100 mM tris-HCl, pH 8.0, 200 mM NH₂OH, neutralised prior to use with KOH, 10 mM ATPm 5 mM MgCl₂, and 30 mM L-aspartate. The control assays contained all the ingredients except L-aspartate. The reactions were performed at 37° C. for 1 h and then terminated by addition of 0.25 ml of a 1:1:1 solution of 10 (w/v) FeCl₃ in 0.1 N HCl, 3N HCl and 12% TCA mixed prior to use. Following 5 min on ice, the reaction mixture was centrifuged (5 min 16,000 g. 4° C. and the light absorbance of the supernatant at 490 nm was measured. For each sample, the non-specific activity obtained in the absence of L-aspartate was substracted from that obtained in its presence.

### EXAMPLE 5

### Analysis of DHPS Activity in Transgenic Potato Tubers

Microtubers or mature tubers were homogenized with mortar and pestle in an equal volume of cold 100 mM tris-HCl pH 7.5 containing 2 mM EDTA, 1.4% sodium ascorbate. 1 mM phenylmethylsulphonilfluoride and 0.5 μg/ml leupeptin. Following 5 min centrifugation (16,000 g at 4° C.) the supernatant was collected. DHPS activity was measured using the O-aminobenzaldehyde (O-ABA) method of Yugari and Gilvarg (1965).

### EXAMPLE 6

### Construction of a Double Gene Construct for Transformation of Sugar Beet by Direct Gene Transfer

In order to introduce the chimeric double construct into sugar beet, the chimeric genes had to be inserted into a plasmid vector specific for direct gene transformation of sugar beet. The plasmid pPG5 (Hall et al., 1996) containing a pat gene which encodes phospbinothricin acetyl transferase was digested with Hind III, filled in and digested with Sac1. The binary vector pAAP50 (FIG. 2B) was digested with Xbal, filled in and digested with Sacl. The blunt/Sac1 fragment containing the double construct was isolated and ligated to the blunt/Sacl fragment of the pPG5 plasmid (pAAP60; FIG. 2D).

### EXAMPLE 7

### Introduction of the Chimeric Genes into Sugar Beet

Shoot cultures of a diploid sugar beet (Beta vulgaris L.) breeding line (Bv-NF, Hall et al., 1993) were used for transformation, according to a protocol which was described before (Hall et al., 1996). In this protocol a polyethylene glycol-mediated DNA transfer technique is used which is applied to protoplast populations enriched specifically for a cell type derived from stomatal guard cells. Stably transformed cells were selected on medium supplemented with 200 μl/l bialaphos for 4 weeks. Bialaphos resistant calli were subsequently subcultured on non-selective medium, and after shoot and root generation plants were put in soil and transferred to the greenhouse.

### EXAMPLE 8

### Analysis of Free Lysine, Threonine and Methionine in Transgenic Sugar Beet Plants

Free amino acid levels were analysed in leaf and tap root tissue from transgenic sugar beet plants according to the protocol described in example 3.

### LITERATURE REFERENCES

Ausubel, F. M. et al. (1994) Current protocols in molecular biology, John Wiley & Sons

Benfey, P. N. et al. (1990) EMBO J. 9: 1685-1696.

Black, S. et al. (1955) J.Biol.Chem. 213: 27-38.

Boy, E. et al. (1979) Siochimie 61: 1151-1160.

Brederode, F. T. et al. (1980) Nucl.Acids Res. 8: 2213-2223.

Bright,S. W. J. et al. (1982) Nature 299: 278-279.

Bryan, J. K. (1980). The Biochemistry of Plants (ed. by B. J. Miflin) Vol. 5: 403-452, Academic Press, N.Y.

Cassan, M. et al. (1986), J.Biol.Chem. 261: 1052-1057.

Cattoir-Reynaerts A. et al. (1983) Biochem.Physiol.Pflanzen 178: 81-90.

Coruzzi G. et al. (1984) EMBO J. 3: 1671-1679.

Dekeyser, R. A. et al. (1990) The Plant Cell 2: 591-602

Dotson, S. B. et al. (1990) Planta 182: 546-552.

Falco,S. C. et al. (1995), Bio/Technology 13: 577-582.

Fluhr, R. et al. (1986) EMBO J. 5: 2063-2071.

Frankard, V. et al. (1992) Plant Physiol. 99: 1285-1293.

Galili, G. (1995) The Plant Cell 7: 899-906.

Gallie, D. R. et al. (1987) Nucl.Acids Res. 15: 3257-3273

Glassman, K. F. (1992) Biosynthesis and Mol. Regul. of Amino Acids in Plants (ed. by B. K. Singh et al.): 217-228.

Gordon-Kann, W. J. et al. (1990) The Plant Cell 2: 603-618.

Greve, H. D. et al. (1983) J.Mol.Appl.Genet. 1: 499-511.

Grierson, D. et al. (1986) Nucl.Acids Res. 14: 8595-8603.

Hall, R. D. et al. (1993) Plant Cell Rep. 12: 339-342.

Hall, R. D. et al. (1996) Nature Biotechn. 14: 1133-1138.

Hayashimoto, A. et al. (1990) Plant Physiol. 93: 657-863.

Herbers, K. et al. (1994) Plant Mol. Biol. 26: 73-83.

H.fgen, R. and Willmitzer, L. (1988) Nucl.Acids Res. 16: 9877.

Horsch, R. B. et al. (1985) Science 227: 1229-1231.

Keil, M. et al. (1989) EMBO J. 8: 1323-1330).

Lea, P. J. et al. (1985) Chemistry and Biochemistry of the Amino Acids (ed. by G. C. Barrett): 197-226, London: Chapman and Hall.

Matthews, B. F. et al. (1989) Plant Physiol. 91: 1569-1574.

McElroy, D. et al. (1990) The Plant Cell 2: 163-171.

Mignery, C. A. et al. (1988) Gene 62: 27-44.

Murashige, T. and Skoog, F. (1962) Physiol.Plant. 15: 473-497.

Negrutiu, I. et al. (1984) Theor.Appl.Genet. 6: 11-20.

Pen, J. et al. (1992) Bio/Techn. 10: 292-296.

Perl, A. et al. (1992) Plant Mol.Biol, 19: 815-823.

Richaud, C. et al.(1973) Eur.J.Biochem. 40: 619-629.

Richaud, F. et al. (1986) J.Bacteriol. 166: 297-300.

Sambrook, J. et al. (1989) Molecular Cloning. A laboratory manual. Cold Spring Harbor Laboratory Press.

Schreier, P. H. et al. (1985) EMBO J. 4: 25-32.

Sengupta-Gopalan, C. (1985) Proc.Natl.Acad.Sci.USA 82: 3320-3324.

Shaul, O. and Galili, G. (1992a) Plant J. 2: 203-209.

Shaul, O. and Galili, G. (1992b) Plant Physiol. 100: 1157-1163.

Shaul, O. and Galili, G. (1993) Plant Mol. Biol. 23: 759-768.

Shedlarski, J. G. and Gilvarg, C. (1970) J.Biol.Chem. 245: 1362-1373.

Smeekens, S. et al. (1985a) Nucl.Acids Res. 13: 3179-3194.

Smeekens, S. et al. (1985b) Nature 317: 456-458.

Umbarger, H. E. (1978) Ann.Rev.Biochem. 47: 533-606.

Van den Broeck, G. et al. (1985) Nature 313: 358-363.

Van Engelen, F. A. et al. (1995) Transgenic Research 4: 288-290.

Visser, R. G. F. (1991) Plant Tissue Culture Manual B5 (ed. by K. Lindsey): 1-9, Kluwer Acad. Publishers, The Netherlands.

Wenzler, H. C. et al. (1989) Plant Mol.Biol. 12: 41-50.

Yugari, Y. and Gilvarg, C. (1962) Biochem.Biophys.Acta 62: 612-614.

Yugari, Y. and Gilvarg, C. (1965) J.Biol.Chem. 240: 4710-4716.

1. A chimeric double gene construct which, when incorporated in a plant cell, is capable of producing at least two amino acids via the aspartate family pathway, in particular lysine and threonine and/or methionine, said construct comprising:
(a) a nucleic acid sequence encoding an enzyme having aspartate kinase (AK) activity,
(b) a nucleic acid sequence encoding an enzyme having dihydrodipicolinate synthase (DHPS) activity,
(c) an expression regulating sequence for the encoding sequence (a) operably linked to (a),
(d) an expression regulating sequence for the encoding sequence (b) operably linked to (b),
(e) a nucleic acid sequence encoding a chloroplast transit peptide involved in translocation of the expression product of (a),
(f) a nucleic acid sequence encoding a chloroplast transit peptide involved in translocation of the expression product of (b),
(g) a nucleic acid sequence providing a transcription termination DNA signal for transcription of (a),
(h) a nucleic acid sequence providing a transcription termination DNA signal for transcription (b),
at least one of the nucleic acid sequences (a) and (b) encoding an enzyme having reduced feedback inhibition relative to the corresponding wild type enzyme, said expression regulating sequences (c) and (d) being such that the expression of the AK encoding sequence (a) occurs at a higher rate than that of the DHPS encoding sequence (b), in that sequence (d) regulates expression of sequence (b) at a later stage during plant development than expression of sequence (a) and/or in that sequence (c) comprises a promoter sequence enabling expression in all plant cells or plant organs, and sequence (d) comprises an organ-specific promoter sequence, and wherein sequence (d) enables expression in one or more water-rich plant organs selected from the group consisting of tubers, flowers, stems, fruits, roots and leaves.

2. A gene construct according to claim 1, wherein sequence (d) comprises the tuber-specific class I patatin promoter from Solanum tuberosum, the proteinase inhibitor II promoter from potato, the cathepsin D inhibitor potato promoter, or the fruit-specific promoter from tomato polygalacturonidase gene.

3. A gene construct according to claim 1, wherein sequence (c) comprises a promoter and the omega sequence derived from the coat protein gene of the tobacco mosaic virus, or the alfalfa mosaic virus translational enhancer.

4. A gene construct according to claim 1, wherein sequence (c) comprises the cauliflower mosaic virus (CaMV) 35S promoter.

5. A gene construct according to claim 1, wherein both sequence (c) and sequence (d) comprise inducible promoters, and wherein the induction stimulus is different for the two promoters.

6. A gene construct according to claim 5, wherein one of the inducible promoters is a light inducible promoter.

7. A gene construct according to claim 1 wherein sequence (e) is a nucleic acid sequence derived from the ferredoxin gene coding for targeting of proteins into the chloroplast stroma, a sequence from the plastocyanine gene coding for targeting of proteins into the chloroplast lumen or a sequence of the transit peptide originating from the pea rbcS-3A gene.

8. A gene construct according to claim 1, wherein sequences (a) and (b) are each fused to a transcription termination DNA signal of (g) and (h) respectively, the termination signals (g) and (h) comprising a 3′ transcription termination and a mRNA polyadenylation signal.

9. A gene construct according to claim 1, wherein sequence (g) and/or (h) comprises the termination signals present at the 3′ flanking region of the pea rbcS gene, the bean phaseolin gene, the nopaline synthase gene derived from the Ti plasmid of Agrobacterium tumefaciens or the octopine synthase gene from the Ti plasmid of A. tumefaciens.

10. A gene construct according to claim 1, wherein sequence (b) encodes an enzyme that is less sensitive to inhibition by lysine, and sequence (a) encodes an enzyme that is less susceptible to inhibition by threonine than the corresponding wild type plant enzymes.

11. A gene construct according to claim 1, wherein sequence (a) is the E. coli mutant LysC gene coding for the isoenzyme AK-III, and sequence (b) is the dapA gene from E. coli.

12. An expression vector comprising a chimeric double gene construct according to claim 1.

13. A transgenic plant comprising a double gene construct according to claim 1.

14. A transgenic plant according to claim 13, wherein the plant is capable of transmitting said double gene construct or the elements thereof to a progeny plant.

15. A transgenic plant according to claim 13, wherein the plant is capable of producing both threonine and lysine at levels more than 5 times those of the wild type plant under the same cultivation conditions.

16. A transgenic plant according to claim 13, wherein the plant is capable of producing methionine at levels exceeding that of the wild type plant under the same cultivation conditions.

17. A transgenic plant according to claim 13, wherein threonine and lysine are overproduced in comparison to the wild type plant under the same cultivation conditions in tubers, stems, fruits, roots, flowers or leaves.

18. A transgenic plant according to claim 17, said plant tuber, flower, stem, root, fruit having at least a water content of 80% under normal cultivation conditions for the non-transgenic plant.

19. A transgenic plant according to claim 13, being a potato plant, sugar beet plant or grass.

20. A part of a plant according to claim 13 comprising at least a cell.

21. A part according to claim 20, said part being derived from a tuber, fruit, root, stem, seed or flower or being a tuber, fruit, stem, seed, flower or leaf.

22. A process for obtaining a plant capable of overproducing at least lysine and threonine, comprising introducing the double gene construct of claim 1 into a plant cell, followed by cultivating the plant cell to tissue or a part of a plant or a mature plant under conditions that the regulating sequences (c) and (d) are activated.

23. A process according to claim 22, wherein the gene construct is introduced into a plant cell via transformation.

24. A process according to claim 22, wherein two plants comprising subfragments of said gene construct are crossed.

25. A process according to claim 22, comprising selecting a part of the plant or tissue obtained and growing a subsequent plant from the tissue or from tissue or organs derived from the plant.

26. A transgenic plant comprising an expression vector according to claim 12.

27. A process for obtaining a plant capable of overproducing at least lysine and threonine, comprising introducing a vector according to claim 12 into a plant cell, followed by cultivating the plant cell to tissue or a part of a plant or a mature plant under conditions that the regulating sequences (c) and (d) are activated.

28. A process according to claim 27, wherein the gene construct is introduced into a plant cell via transformation.

29. A process according to claim 27, wherein two plants comprising subfragments of said gene construct are crossed.

30. A process according to claim 27, comprising selecting a part of the plant or tissue obtained and growing a subsequent plant from the tissue or from tissue or organs derived from the plant.
